(19) United States
(12) Patent Application Publication
(10) Pub. No.: US 2008/0046084 A1
(43) Pub. Date: Feb. 21, 2008

(54) ANTERIOR CERVICAL SPINE INSTRUMENTATION AND RELATED SURGICAL METHOD

(76) Inventor: John Sledge, (US)

Correspondence Address: CLEMENTS WALKER, 1901 ROXBOROUGH ROAD, SUITE 300, CHARLOTTE, NC, 28211, US

(21) Appl. No.: 11/842,617

(22) Filed: Aug. 21, 2007

Related U.S. Application Data
(60) Provisional application No. 60/839,095, filed on Aug. 21, 2006.

(51) Int. Cl. A61F 2/44; A61B 17/58

(52) U.S. Cl. 623/17.16; 606/90; 606/96

(57) ABSTRACT

In various exemplary embodiments, the present invention provides a set of less invasive cervical spine instruments that are used to achieve cervical disc decompression, bone preparation, and the alignment of one or more matched sized bone grafts prior to cervical plate placement. This set of less invasive cervical spine instruments, and the related surgical method, result in reduced surgical time, the preparation of a precise machined bone surface while simultaneously maintaining the cervical disc decompression height of the intervertebral endplates, the selection of one or more prefabricated bone dowel grafts sized to match the machined bone surface and maximizing the surface contact required for cervical spine fusion, the placement of the one or more prefabricated bone dowel grafts (e.g. side by side) that can be of different diameters in order to fully exploit the intervertebral space available, and the alignment of the cervical plate using a cervical spine instrument that is matched to align with the one or more prefabricated bone dowel grafts beneath the cervical plate.

### CROSS-REFERENCE TO RELATED APPLICATION(S)

The present non-provisional patent application claims the benefit of priority of U.S. Provisional Patent Application No. 60/839,095 (John SLEDGE), filed on Aug. 21, 2006, and entitled “ANTERIOR CERVICAL SPINE INSTRUMENTATION AND RELATED SURGICAL METHOD,” the contents of which are incorporated in full by reference herein.

### FIELD OF THE INVENTION

The present invention relates generally to anterior cervical spine instrumentation and a related surgical method for aligning a cervical plate over an intervertebral bone graft. More specifically, the present invention relates to a set of less invasive cervical spine instruments that can be used to achieve cervical disc decompression, bone preparation, and the alignment of one or more matched sized bone grafts prior to cervical plate placement.

### BACKGROUND OF THE INVENTION

A large portion of conventional surgical methods for cervical spine fusion is dedicated to performing cervical disc decompression, maintaining a desired cervical disc decompression height, and fine tuning the bone quality of the intervertebral endplates such that they adequately accept a properly matched bone graft or cage. Conventional surgical instruments for cervical spine fusion include the Anterior Lumbar Internal Fixation (ALIF) system (Zimmer, Inc.) which consists of a plurality of distractors and retractors, as well as several other retractors and drill guides. No conventional surgical instruments for cervical spine fusion are known in the art, however, that use a retractor in combination with a drill guide as part of an integrated method for bone graft selection and placement.

Thus, what is needed in the art is a set of less invasive cervical spine instruments that can be used to achieve cervical disc decompression, bone preparation, and the alignment of one or more matched sized bone grafts prior to cervical plate placement. This set of less invasive cervical spine instruments, and the related surgical method, would ideally result in reduced surgical time, the preparation of a precise machined bone surface while simultaneously maintaining the cervical disc decompression height of the intervertebral endplates, the selection of one or more prefabricated bone dowel grafts sized to match the machined bone surface and maximizing the surface contact required for cervical spine fusion, the placement of the one or more prefabricated bone dowel grafts (e.g. side by side) that can be of different diameters in order to fully exploit the intervertebral space available, and the alignment of the cervical plate using a cervical spine instrument that is matched to align with the one or more prefabricated bone dowel grafts beneath the cervical plate.

### BRIEF SUMMARY OF THE INVENTION

In various exemplary embodiments, the present invention provides a set of less invasive cervical spine instruments that are used to achieve cervical disc decompression, bone preparation, and the alignment of one or more matched sized bone grafts prior to cervical plate placement. This set of less invasive cervical spine instruments, and the related surgical method, result in reduced surgical time, the preparation of a precise machined bone surface while simultaneously maintaining the cervical disc decompression height of the intervertebral endplates, the selection of one or more prefabricated bone dowel grafts sized to match the machined bone surface and maximizing the surface contact required for cervical spine fusion, the placement of the one or more prefabricated bone dowel grafts (e.g. side by side) that can be of different diameters in order to fully exploit the intervertebral space available, and the alignment of the cervical plate using a cervical spine instrument that is matched to align with the one or more prefabricated bone dowel grafts beneath the cervical plate.

The set of less invasive cervical spine instruments is based on an alignment guide feature that is built into a soft tissue retractor (spreader). The placement and positioning of all subsequent instruments are constrained by this alignment guide feature, thereby allowing for the preparation of a precise machined bone surface, the placement of one or more prefabricated bone dowel grafts, and the alignment of the cervical plate that is matched to align with the one or more prefabricated bone dowel grafts beneath the cervical plate.

In one exemplary embodiment, the present invention provides a set of cervical spine instruments for achieving cervical disc decompression, bone preparation, and the alignment of one or more matched sized bone grafts prior to cervical plate placement, the set of cervical spine instruments including: a retractor device including a front member defining an opening and a pair of opposed wing members each having structured top and bottom edges for engaging bone and preventing expulsion of the retractor device, wherein each of the pair of opposed wing members includes one or more guide channels; and one or more dilator devices configured to pass through the opening defined by the front member of the retractor device; wherein each of the one or more dilator devices includes one or more guide protrusions configured to engage the one or more guide channels of the pair of opposed wing members of the retractor device. Each of the one or more dilator devices also includes a shoulder stop configured to engage the front member of the retractor device. The set of cervical spine instruments also includes a drill guide defining one or more substantially circular internal channels that are each configured to receive a drill bit, wherein the drill guide includes one or more guide protrusions configured to engage the one or more guide channels of the pair of opposed wing members of the retractor device. The drill guide also includes a shoulder stop configured to engage the front member of the retractor device. The one or more substantially circular internal channels of the drill guide are arranged in one of a single-channel configuration, a dual-channel configuration, and a dual overlapping-channel configuration. The set of cervical spine instruments further includes one or more bone graft dowel pins that are configured to pass through the opening defined by the front member of the retractor device, wherein each of the one or more bone graft dowel pins includes a reference hole at its end. The set of cervical spine instruments still further includes a cervical plate configured to be disposed over the one or more done graft dowel pins once they are placed, wherein the cervical plate includes one or more screw holes disposed therethrough and one or more holes or slots disposed therethrough for receiving one or more alignment pins that are each configured to engage the reference hole of each of the one or more bone graft dowel pins.

In another exemplary embodiment, the present invention provides a surgical method for achieving cervical disc decompression, bone preparation, and the alignment of one or more matched sized bone grafts prior to cervical plate placement, the surgical method including: placing a smaller retractor device between adjacent intervertebral enplates of the spine of a patient, the smaller retractor device including a front member defining an opening and a pair of opposed wing members each having structured top and bottom edges for engaging bone and preventing expulsion of the retractor device, wherein each of the pair of opposed wing members includes one or more guide channels; disposing one or more successively larger dilator devices through the opening defined by the front member of the retractor device and into the intervertebral space between the adjacent intervertebral endplates; wherein each of the one or more dilator devices includes one or more guide protrusions configured to engage the one or more guide channels of the pair of opposed wing members of the smaller retractor device; and placing a larger retractor device between the adjacent intervertebral enplates of the spine of the patient, the larger retractor device including a front member defining an opening and a pair of opposed wing members each having structured top and bottom edges for engaging bone and preventing expulsion of the retractor device, wherein each of the pair of opposed wing members includes one or more guide channels. Each of the one or more successively larger dilator devices also includes a shoulder stop configured to engage the front member of the smaller retractor device. The surgical method also includes disposing a portion of a drill guide defining one or more substantially circular internal channels that are each configured to receive a drill bit through the opening defined by the front member of the larger retractor device, wherein the drill guide includes one or more guide protrusions configured to engage the one or more guide channels of the pair of opposed wing members of the larger retractor device. The drill guide also includes a shoulder stop configured to engage the front member of the larger retractor device. The one or more substantially circular internal channels of the drill guide are arranged in one of a single-channel configuration, a dual-channel configuration, and a dual overlapping-channel configuration. The surgical method further includes disposing one or more bone graft dowel pins through the opening defined by the front member of the retractor device, wherein each of the one or more bone graft dowel pins includes a reference hole at its end. The surgical method still further includes disposing a cervical plate over the one or more done graft dowel pins once they are placed, wherein the cervical plate includes one or more screw holes disposed therethrough and one or more holes or slots disposed therethrough for receiving one or more alignment pins that are each configured to engage the reference hole of each of the one or more bone graft dowel pins.

### BRIEF DESCRIPTION OF THE DRAWINGS

The present invention is illustrated and described herein with reference to the various drawings, in which like reference numbers are used to denote like instrumentation components/parts and/or method steps, as appropriate, and in which:

FIG. 1a is a perspective view of one exemplary embodiment of the retractor (spreader) of the present invention;

FIG. 1b is a front planar view of one exemplary embodiment of the retractor (spreader) of the present invention;

FIG. 1c is a side planar view of one exemplary embodiment of the retractor (spreader) of the present invention;

FIG. 2 is a side planar view of one exemplary embodiment of the dilator of the present invention, illustrated engaging the retractor of FIG. 1;

FIG. 3 is a perspective view of one exemplary embodiment of the drill guide of the present invention, illustrated engaging the retractor of FIG. 1;

FIG. 4a is a perspective view of one exemplary embodiment of the drill guide of the present invention;

FIG. 4b is a side planar view of one exemplary embodiment of the drill guide of the present invention;

FIG. 4c is a front planar view of one exemplary embodiment of the drill guide of the present invention;

FIG. 5 is a perspective view of one exemplary embodiment of the bone graft dowel pin of the present invention;

FIG. 6a is a perspective view of one exemplary embodiment of the cervical plate of the present invention;

FIG. 6b is a front planar view of one exemplary embodiment of the cervical plate of the present invention;

FIG. 6c is a side planar view of one exemplary embodiment of the cervical plate of the present invention;

FIG. 7 is a perspective view of one exemplary embodiment of the cervical plate screw of the present invention; and

FIG. 8 is a schematic diagram illustrating a dual overlapping channel configuration associated with the drill guide of FIG. 4.

### DETAILED DESCRIPTION OF THE INVENTION

In various exemplary embodiments, the present invention provides a set of less invasive cervical spine instruments that are used to achieve cervical disc decompression, bone preparation, and the alignment of one or more matched sized bone grafts prior to cervical plate placement. This set of less invasive cervical spine instruments, and the related surgical method, result in reduced surgical time, the preparation of a precise machined bone surface while simultaneously maintaining the cervical disc decompression height of the intervertebral endplates, the selection of one or more prefabricated bone dowel grafts sized to match the machined bone surface and maximizing the surface contact required for cervical spine fusion, the placement of the one or more prefabricated bone dowel grafts (e.g. side by side) that can be of different diameters in order to fully exploit the intervertebral space available, and the alignment of the cervical plate using a cervical spine instrument that is matched to align with the one or more prefabricated bone dowel grafts beneath the cervical plate.

The set of less invasive cervical spine instruments is based on an alignment guide feature that is built into a soft tissue retractor (spreader). The placement and positioning of all subsequent instruments are constrained by this alignment guide feature, thereby allowing for the preparation of a precise machined bone surface, the placement of one or more prefabricated bone dowel grafts, and the alignment of the cervical plate that is matched to align with the one or more prefabricated bone dowel grafts beneath the cervical plate.

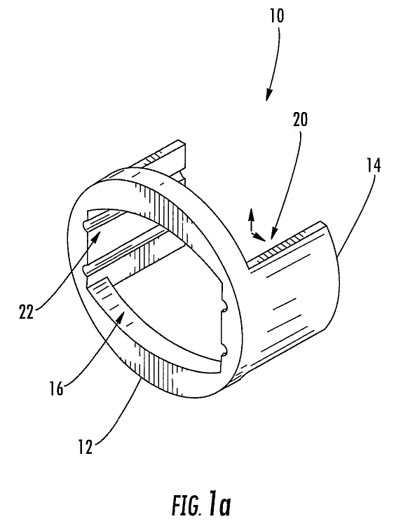

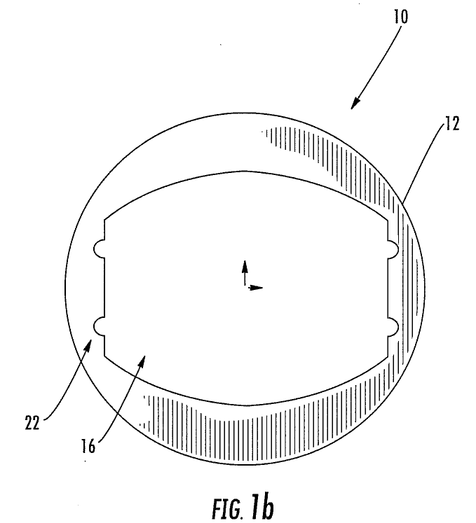

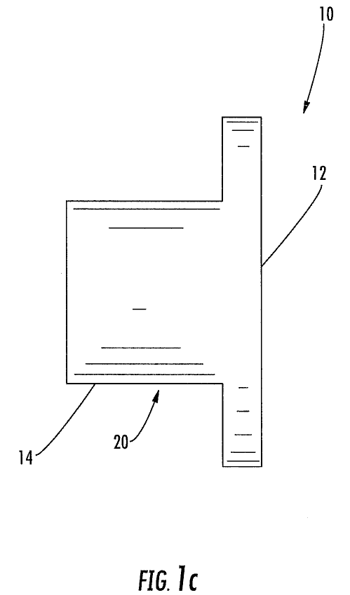

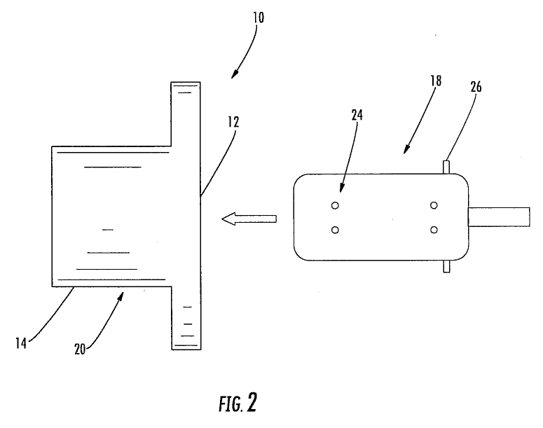

Referring to FIG. 1, in one exemplary embodiment of the present invention, the set of cervical spine instruments includes a retractor (spreader) 10 having a front member 12 and a pair of opposed wing members 14 (FIGS. 1a and 1c). The front member 12 of the retractor 10 defines an opening 16 (FIGS. 1a and 1b) that is configured to receive a dilator 18 (FIG. 2) of a series of dilators. Preferably, the pair of opposed wing members 14 each have serrated or otherwise structured top and bottom edges 20 (FIGS. 1a and 1c) that are designed to engage bone and prevent expulsion when the retractor 10 is placed between intervertebral endplates, for example. Preferably, the pair of opposed wing members 14 also each include a pair of internal guide channels 22 (FIGS. 1a and 1b) that are configured to receive one or more pairs of corresponding external guide protrusions 24 (FIG. 2) associated with the dilator 18 of the series of dilators. As described above, the internal guide channels 22 and external guide protrusions 24 assist in the placement and positioning of the dilator 18 of the series of dilators and all subsequent instruments having external guide protrusions 24, holding these instruments straight and stable within the retractor 10. In general, the retractor 10 has overall front dimensions on the order of 8-16 mm and overall side dimensions on the order of 6-12 mm. In general, the series of dilators have overall dimensions on the order of 6-20 mm. For example, the dilator 18 of the series of dilators has two preferred widths 14 mm and 18 mm. The dilator 18 of the series of dilators comes in graduated thicknesses ranging from 5 mm to 12 mm, for example. These graduated dilators are inserted into the retractor 10 sequentially in order to distract and decompress the excised intervertebral space. The retractor 10 is wedged into the excised intervertebral space when attached to the proper sized dilator 18 of the series of dilators, which is subsequently removed. Optionally, the dilator 18 of the series of dilators includes a pair of opposed end stops 26 (FIG. 2) that are configured to constrain and limit the insertion of the dilator 18 of the series of dilators into (and through) the opening 16 of the front member 12 of the retractor 10.

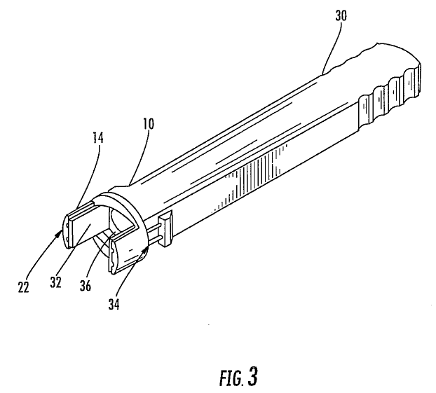

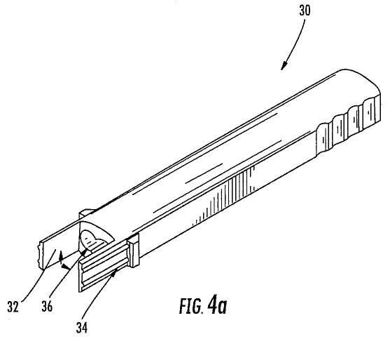

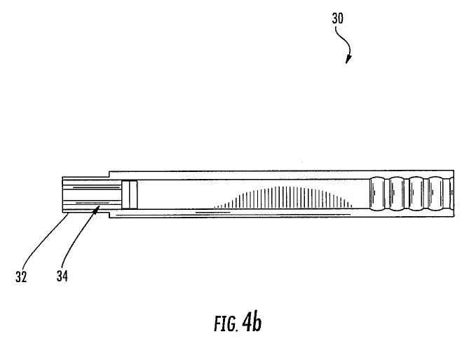

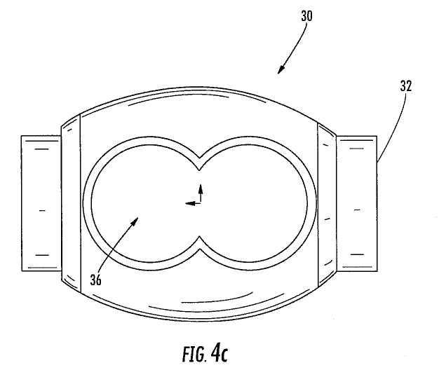

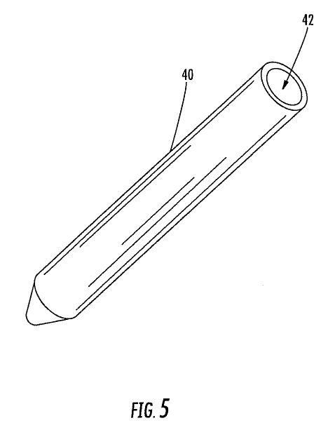

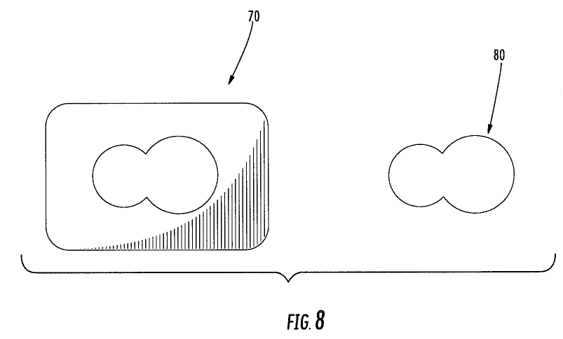

Referring to FIG. 3, in one exemplary embodiment of the present invention, the set of cervical spine instruments also includes a drill guide 30 that engages the retractor 10. Specifically, a pair of opposed wing members 32 of the drill guide 30 engage the retractor 10 via the pair of internal guide channels 22 of each of the pair of opposed wing members 14 associated with the retractor 10 and one or more pairs of corresponding external guide protrusions 34 of the opposed wing members 32 associated with the drill guide 30, holding the drill guide 30 straight and stable within the retractor 10. The drill guide 30 defines one or more substantially circular internal channels 36 (FIGS. 3, 4a, and 4c) that are configured to receive a drill having a shoulder stop (not illustrated). The drill having the shoulder stop is used to ream the intervertebral endplates using a substantially circular drill pattern. Various drill guides having various channel diameters and configurations can be used. For example, a single-channel configuration can be used, a dual-channel configuration can be used, and a dual overlapping-channel configuration can be used. In the event that channels having different diameters are desired, subsequently placed drill guides can also be employed. The resulting drill pattern is plugged using one or more bone graft dowel pins 40 (FIG. 5) or the like. In the case of a dual overlapping channel configuration, for example, the first hole is drilled, the first bone graft dowel pins 40 is placed, the second hole is drilled (including a portion of the first bone graft dowel pin), and the second bone graft dowel pin 40 is placed. The related cutting jig 70 and resulting bone graft placement 80 are illustrated in FIG. 8. Each bone graft dowel pin 40 includes a reference hole 42, the function of which is described in greater detail herein below.

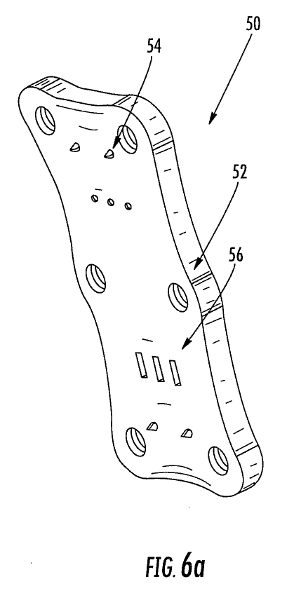

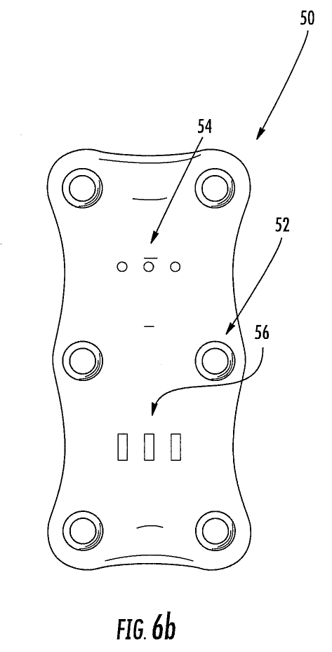

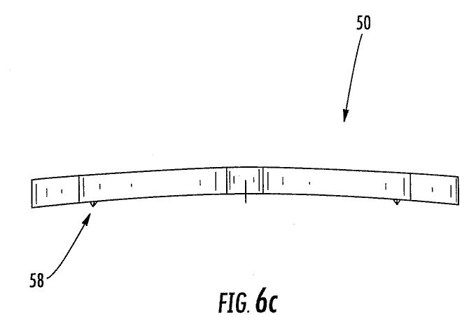

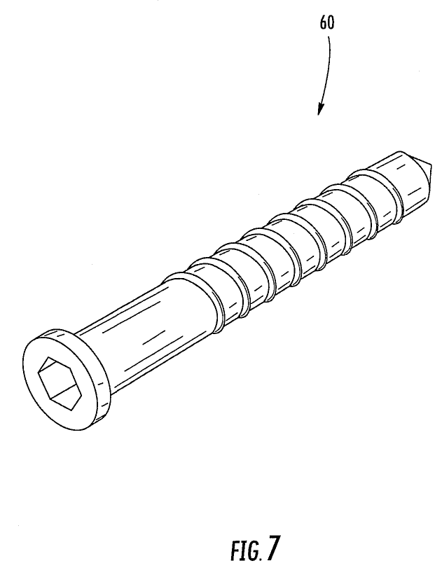

Referring to FIG. 6, in one exemplary embodiment of the present invention, the set of cervical spine instruments further includes a cervical plate 50 that is configured to cover and secure the one or more bone graft dowel pins 40 (FIG. 5) after they are placed. The cervical plate 50 includes a plurality of cervical plate screw holes 52 that are configured to receive a plurality of cervical plate screws 60 (FIG. 7), well known to those of ordinary skill in the art. The cervical plate 50 also includes a plurality of alignment holes 54 and alignment slots 56 that are configured to selectively receive a plurality of alignment pins 58 (FIG. 6c). Preferably, the alignment pins 58 protrude slightly beyond (or above) the interior (or back) surface of the cervical plate 50, such that they engage the reference hole(s) 42 (FIG. 5) of the bone graft dowel pin(s) 40 (FIG. 5), thereby temporarily aligning the cervical plate 50 with respect to the bone graft dowel pin(s) 40 until it can be permanently affixed using the cervical plate screws 60. Advantageously, the alignment slots 56 permit this flexible procedure to be used in cases in which varied spacing or multiple levels are involved. At the completion of this flexible procedure, the bone graft dowel pin(s) 40 are properly positioned and secured within the intervertebral space. It should be noted that one or more cervical plate holding instruments (not illustrated) or the like can also be used.

Regarding the use of the set of cervical spine instruments of the present invention, a standard surgical approach to the anterior neck (or back) is performed. This approach can be undertaken from either the left or the right. Deep retractors, of a type and number conventionally used, are placed. Once the anterior aspect of the cervical (or lumbar) spine has been exposed, and the anterior longitudinal ligament, most of a disc and the anterior osteophytes are removed. The disc dissection is taken out to the Joints of Lushka/Unco-vertebral joints to maximize disc exposure and potential graft surface. An undersized spreader is then placed into the space to check width and height. Sequentially larger (in height) dilators are placed into the disc space until the desired lordosis is reconstituted. A spreader of a matching height and width is selected. Two widths of spreaders are preferred so as to have a wide one (18 mm OD) and a narrow one (14 or 16 mm OD). The dilator is then inserted into the spreader and it is tapped into place between the vertebrae holding the spreader. Standard decompression is then performed with the spreader in place using the usual instruments. Once the decompression is complete, the surgeon moves forward to complete the remainder of the procedure, which includes the preparation of the graft bed, placement of the graft, and placement of the plate.

The size of the spreader placed is known so the width of the drill guide is predetermined and the minimum height is known. The height of the graft to be placed is determined by the amount of bone that is removed during the decompression. This height can be guessed at or measured with calipers. The goal is bone contact along the entire length of the graft. It may be that a 6 mm graft is needed on one side and an 8 mm graft is needed on the other. This is not a problem, but necessitates changing drill guides after the first graft is placed prior to placing the second one.

The chosen drill guide fits into the spreader and uses it and/or its contact against the anterior cortex and/or the flanges that go down into the disc space as an alignment guide and for stabilization. The groves in the spreader are at a fixed distance so any of the drill guides can be placed into any of the spreaders. The location of the spreader determines the position of the graft; while the position of the graft determines the size and location of the plate.

For the grafts, the smaller sizes typically use two plugs, the larger sizes typically use one plug. The drill guide either provides one central hole or two adjacent holes. The centers of the two graft systems all have the same intergraft spacing; therefore, the distance between the centers of the graft is the same for 6, 7, 8, or 9 mm grafts. If the narrow spreader is chosen then the grafts will switch to a single graft at a smaller diameter. When the larger double grafts are used one hole is drilled and the graft is placed and then the second hole is drilled and the second graft is placed. In drilling the second hole a portion of the first graft will be in the path of the drill and will be removed by the drill. The drill guide has a flat top and the drill has an adjustable depth set for a positive stop at 14 mm or 16 mm. The graft inserter has a similarly adjustable depth set. The drilling and placement of the graft are both done with the drill guide in place.

For the graft, dense cancellous bone is preferred; threaded, if possible, to increase the surface area and the rigidity of the construct. If threaded, the hole is undersized by 1 mm and there is the additional step of tapping the hole. If non-threaded, a line to line fit between the drill and the graft is sufficient. The bone plugs are then inserted into the milled holes with the insertion device. With the decompression performed and the graft in place it is time to remove the spreader so that the plate can be applied. A removal tool fits over the lateral margin of the spreader and locks in place to allow for the removal of the spreader. Flanges on the side of the removal device are hinged so that they can widen to fit over the edges of the spreader. The lever arm is spring loaded so that the flanges will spread and then snap into place once over the outside lip of the spreader. The lip on the spreader is angled anteriorly towards the midline so as the connection becomes more stable as it is put under tension. A rotational tightening mechanism locks the removal tool in place if required by rotating the inner handle 90 degrees. This forces an inclined piece under the medial end of the lever arm and locks the distraction tool in place over the spreader.

The plates have three designators: 1. a number indicating the true length of the plate; 2. a number associated with the size graft that the plate will fit over; and 3. a letter indicating narrow or wide. A plate that is listed as a 22/10/W plate is 22 mm in overall length, will appropriately span a 10 mm graft, and is wide. To span a 10 mm graft, the tread-to-tread screw distance is about 14 mm. Different size grafts can be placed side-by-side at the same level by changing the drill guide between drillings. If the two grafts used at the same level are 8 mm and 9 mm, respectively, then a plate with at least a 9 as the middle number is chosen to appropriately span the graft. Both grafts will have the same center as determined by their respective drill guides.

Again, all bone plugs have a small central hole, with a flange, if threaded, for the insertion device and for the positioning pins on the plates. The plates have 3 holes to accept holding pins to center the plate over the bone plugs. If two grafts are used then two pins are used. If one central graft is used then one pin is used. The pins go through the plate and friction lock. They have a blunt tip to facilitate engagement into the hole of the graft. They extend above the plate and can be grabbed, to be used as a plate positioner.

For single level procedures, the plate is centered over the graft(s) with the guide pins going into the holes of the grafts. This automatically aligns the plate rostrally/caudally, side-to-side, and rotationally. Small spikes are disposed on the back of plate and the plates are pre-bent. The plate is then tapped with a tamp to seat the small spikes on the back of the plate. These may not be needed in every case as the guide pins may adequately perform the same function. After the four screws are placed, the guide pins are removed.

Multi-level plate insertion requires an extra step. After the grafts have been placed at either two levels or three levels, calipers are used to determine the distance between the center holes of the grafts placed at either end. This yields a distance that is denoted by a scaling letter read from the calipers. This letter and the size of the largest graft used are entered into a box chart that is printed on the instrument tray. This yields a number that is analogous to the graft size used for plate selection for single levels. Using this number, a plate is selected that has at least this digit as the center number for the plate. For multi-level plates, one end has the standard three holes for temporary holding pins and the other end has slots to be used if desired. The screws are then placed and the pins are removed. In this manner, if the instruments are used in an integrated fashion, one step will lead to another step.

Although the present invention has been illustrated and described herein with reference to preferred embodiments and specific examples thereof, it will be readily apparent to those of ordinary skill in the art that other embodiments and examples may perform similar functions and/or achieve like results. All such equivalent embodiments and examples are within the spirit and scope of the present invention, are contemplated thereby, and are intended to be covered by the following claims.

What is claimed is:

1. A set of cervical spine instruments for achieving cervical disc decompression, bone preparation, and the alignment of one or more matched sized bone grafts prior to cervical plate placement, the set of cervical spine instruments comprising:
a retractor device comprising a front member defining an opening and a pair of opposed wing members each having structured top and bottom edges for engaging bone and preventing expulsion of the retractor device, wherein each of the pair of opposed wing members comprises one or more guide channels; and
one or more dilator devices configured to pass through the opening defined by the front member of the retractor device; wherein each of the one or more dilator devices comprises one or more guide protrusions configured to engage the one or more guide channels of the pair of opposed wing members of the retractor device.

2. The set of cervical spine instruments of claim 1, wherein each of the one or more dilator devices further comprises a shoulder stop configured to engage the front member of the retractor device.

3. The set of cervical spine instruments of claim 1, further comprising a drill guide defining one or more substantially circular internal channels that are each configured to receive a drill bit, wherein the drill guide comprises one or more guide protrusions configured to engage the one or more guide channels of the pair of opposed wing members of the retractor device.

4. The set of cervical spine instruments of claim 3, wherein the drill guide further comprises a shoulder stop configured to engage the front member of the retractor device.

5. The set of cervical spine instruments of claim 3, wherein the one or more substantially circular internal channels of the drill guide are arranged in one of a single-channel configuration, a dual-channel configuration, and a dual overlapping-channel configuration.

6. The set of cervical spine instruments of claim 1, further comprising one or more bone graft dowel pins that are configured to pass through the opening defined by the front member of the retractor device, wherein each of the one or more bone graft dowel pins comprises a reference hole at its end.

7. The set of cervical spine instruments of claim 6, further comprising a cervical plate configured to be disposed over the one or more done graft dowel pins once they are placed, wherein the cervical plate comprises one or more screw holes disposed therethrough and one or more holes or slots disposed therethrough for receiving one or more alignment pins that are each configured to engage the reference hole of each of the one or more bone graft dowel pins.

8. A set of cervical spine instruments for achieving cervical disc decompression, bone preparation, and the alignment of one or more matched sized bone grafts prior to cervical plate placement, the set of cervical spine instruments comprising:
a retractor device comprising a front member defining an opening and a pair of opposed wing members each having structured top and bottom edges for engaging bone and preventing expulsion of the retractor device, wherein each of the pair of opposed wing members comprises one or more guide channels;
one or more dilator devices configured to pass through the opening defined by the front member of the retractor device; wherein each of the one or more dilator devices comprises one or more guide protrusions configured to engage the one or more guide channels of the pair of opposed wing members of the retractor device; and
a drill guide defining one or more substantially circular internal channels that are each configured to receive a drill bit, wherein the drill guide comprises one or more guide protrusions configured to engage the one or more guide channels of the pair of opposed wing members of the retractor device.

9. The set of cervical spine instruments of claim 8, wherein each of the one or more dilator devices further comprises a shoulder stop configured to engage the front member of the retractor device.

10. The set of cervical spine instruments of claim 8, wherein the drill guide further comprises a shoulder stop configured to engage the front member of the retractor device.

11. The set of cervical spine instruments of claim 8, wherein the one or more substantially circular internal channels of the drill guide are arranged in one of a single-channel configuration, a dual-channel configuration, and a dual overlapping-channel configuration.

12. The set of cervical spine instruments of claim 8, further comprising one or more bone graft dowel pins that are configured to pass through the opening defined by the front member of the retractor device, wherein each of the one or more bone graft dowel pins comprises a reference hole at its end.

13. The set of cervical spine instruments of claim 12, further comprising a cervical plate configured to be disposed over the one or more done graft dowel pins once they are placed, wherein the cervical plate comprises one or more screw holes disposed therethrough and one or more holes or slots disposed therethrough for receiving one or more alignment pins that are each configured to engage the reference hole of each of the one or more bone graft dowel pins.

14. A surgical method for achieving cervical disc decompression, bone preparation, and the alignment of one or more matched sized bone grafts prior to cervical plate placement, the surgical method comprising:
placing a smaller retractor device between adjacent intervertebral enplates of the spine of a patient, the smaller retractor device comprising a front member defining an opening and a pair of opposed wing members each having structured top and bottom edges for engaging bone and preventing expulsion of the retractor device, wherein each of the pair of opposed wing members comprises one or more guide channels;
disposing one or more successively larger dilator devices through the opening defined by the front member of the retractor device and into the intervertebral space between the adjacent intervertebral endplates; wherein each of the one or more dilator devices comprises one or more guide protrusions configured to engage the one or more guide channels of the pair of opposed wing members of the smaller retractor device; and
placing a larger retractor device between the adjacent intervertebral enplates of the spine of the patient, the larger retractor device comprising a front member defining an opening and a pair of opposed wing members each having structured top and bottom edges for engaging bone and preventing expulsion of the retractor device, wherein each of the pair of opposed wing members comprises one or more guide channels.

15. The surgical method of claim 14, wherein each of the one or more successively larger dilator devices further comprises a shoulder stop configured to engage the front member of the smaller retractor device.

16. The surgical method of claim 14, disposing a portion of a drill guide defining one or more substantially circular internal channels that are each configured to receive a drill bit through the opening defined by the front member of the larger retractor device, wherein the drill guide comprises one or more guide protrusions configured to engage the one or more guide channels of the pair of opposed wing members of the larger retractor device.

17. The surgical method of claim 16, wherein the drill guide further comprises a shoulder stop configured to engage the front member of the larger retractor device.

18. The surgical method of claim 16, wherein the one or more substantially circular internal channels of the drill guide are arranged in one of a single-channel configuration, a dual-channel configuration, and a dual overlapping-channel configuration.

19. The surgical method of claim 14, disposing one or more bone graft dowel pins through the opening defined by the front member of the retractor device, wherein each of the one or more bone graft dowel pins comprises a reference hole at its end.

20. The surgical method of claim 19, further comprising disposing a cervical plate over the one or more done graft dowel pins once they are placed, wherein the cervical plate comprises one or more screw holes disposed therethrough and one or more holes or slots disposed therethrough for receiving one or more alignment pins that are each configured to engage the reference hole of each of the one or more bone graft dowel pins.
